(19) United States
(12) Patent Application Publication
(10) Pub. No.: US 2006/0239951 A1
(43) Pub. Date: Oct. 26, 2006

(54) Methods for stimulating hair growth by administering BMPs

(76) Inventors: Alexandre Valentin, Belmont, MA (US); John Wozney, Hudson, MA (US); Edward J. Kilbourne, Hopewell, NJ (US); Kieron Leslie, Norfolk (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/393,498

(22) Filed: Mar. 30, 2006

Related U.S. Application Data
(60) Provisional application No. 60/666,172, filed on Mar. 30, 2005.

(51) Int. Cl. A61K 8/64; A61K 38/18

(52) U.S. Cl. 424/70.14; 514/12

(57) ABSTRACT

Methods and compositions for stimulating hair growth and inhibiting immune system activity by administering BMPs are provided. The methods and compositions can be used for treating or preventing disorders resulting in loss of hair, as well as a wide range of autoimmune disorders.

This application claims priority to U.S. Patent Application No. 60/666,172, filed on Mar. 30, 2005, which is herein incorporated by reference in its entirety.

### FIELD OF THE INVENTION

The present invention relates to methods of stimulating hair growth, and to pharmaceutical compositions that stimulate hair growth. The present invention also relates to methods of inhibiting the immune system, and to pharmaceutical compositions that inhibit the immune system.

### BACKGROUND OF THE INVENTION

Tens of millions of Americans suffer from some type of hair loss. A wide variety of conditions cause hair loss, including androgenic alopecia, or common pattern baldness, anagen effluvium a chemotherapy-induced hair loss, telogen effluvium, induced by stress, fever and drugs and alopecia areata, an autoimmune disease which afflicts an estimated four million people. Cotsarelis et al., “Towards a molecular understanding of hair loss and its treatment,” TRENDS in Mol. Med. 7(7):293-301 (2001); NATIONAL INSTITUTES OF HEALTH, Questions & Answers About Alopecia Areata (2003); MacDonald, N., “Alopecia areata: identification and current treatment approaches,” Dermatol. Nurs. 11:356-359 (1999). While not life-threatening, hair-related disorders affect personal appearance, and therefore frequently have profound impact on patients' social interactions, self-esteem, and psychological well being.

Androgenic alopecia affects both men and women, although women tend to lose less hair, and in a more diffuse pattern than men. There is also evidence that androgenic hormones, such as testosterone, coupled with a genetic predisposition, are necessary for the development of male pattern baldness. It is currently believed that the conversion of testosterone into dihydrotestosterone, a compound which inhibits hair growth, by the enzyme 5-α-reductase, triggers pattern baldness in men, but the mechanism of interaction between the hormone and hair follicles remains unknown. Female pattern baldness is thought to result from a decrease in estrogen, a hormone that normally counteracts the balding effect of testosterone, although there is so far no consensus on whether pattern baldness in women is truly androgen-dependent. Vierhapper et al., “Production rates of testosterone and of dihydrotestosterone in female pattern hair loss,” Metabolism 52(7):927-929 (2003).

Telogen effluvium manifests as excessive shedding of hair, which occurs as cycling hair follicles prematurely enter the resting phase of the hair growth cycle, called telogen. It may be precipitated by a multitude of stress-related causes, including high fevers, childbirth, severe infections, severe chronic illness, severe psychological stress, major surgery, an over- or under-active thyroid gland, crash diets with inadequate protein, and a variety of medications, including, e.g., retinoids, beta blockers, calcium channel blockers, antidepressants, and non-steroidal anti-inflammatories, including ibuprofen and acetominophen. Generally little treatment is possible beyond identifying and either treating or discontinuing the inciting factor, whichever is appropriate. In most cases, the lost hair will be replaced within six to twelve months. Paus, et al., “The biology of hair follicles,” N. Engl. J. Med. 341(7):491-497 (1999).

Most cases of drug-induced alopecia involve normal hairs entering telogen prematurely, as in telogen effluvium. In contrast, anagen effluvium, the most common type of chemotherapy-induced alopecia, results from the abrupt cessation of mitotic activity in hair matrix cells of anagen hair follicles. This induces the follicles to produce either no hair, or produce only narrow defective hair sheaths which are predisposed to fracture and loss. This type of alopecia can be seen to some degree in most anti-neoplastic therapies, depending on dosage and route of administration. However, there are certain agents, such as bleomycin, cisplatin, doxorubicin, vinblastine and vincristine, which induce alopecia more frequently and severely. These agents display a synergistic effect when used in combination and may cause severe and complete alopecia. Anagen effluvium manifests within 1 to 2 weeks after the beginning of chemotherapy but is most noticeable 1 to 2 months later. Initially, there may not be total hair loss, since approximately 10% of follicles will not be in anagen phase at the start of chemotherapy. Total hair loss eventually occurs with prolonged therapy, which can also induce hair loss in other areas of the body. Hair regrowth can usually be expected after the end of chemotherapy, although hair color and texture may change.

Alopecia areata usually presents as varying amounts of patchy hair loss, most commonly on the scalp (though it can affect any hair-bearing surface), but may also manifest as larger patches with little or no hair. Related forms of the disease include: (1) alopecia totalis, characterized by complete loss of all scalp hair; and (2) alopecia universalis, characterized by loss of all body hair, including eyelashes, eyebrows, underarm hair, and pubic hair. The latter form can cause serious respiratory problems because the nostrils and sinuses are no longer protected from airborne foreign particles. Hull et al., “Guidelines for the management of alopecia areata,” Brit. J. Dermatol. 149:692-699 (2003).

Alopecia areata is an autoimmune disease in which cells of the anagen hair bulb are attacked by T lymphocytes. In a process resembling chemotherapy-induced hair loss, lymphocyte infiltration of the growing hair bulb forces the anagen follicles into dystrophic catagen, causing the hair shaft to break off. Possible targets of autoimmune attack in alopecia areata include matrix keratinocytes, dermal papilla cells, and melanocytes. Cotsarelis et al., supra. Linkage analyses indicate that this disease has a genetic component, though the range of associated genes, including the major histocompatibility complex, cytokine and immunoglobulin genes, suggests that any genetic predisposition is likely multifactorial. Hull et al., supra. In any case, whether the underlying defect in alopecia areata lies within the hair follicle, the immune system, or both, is not known. Kalish, et al., “Alopecia areata: autoimmunity—the evidence is compelling,” J. Invest. Dermatol. 8(2):164-167 (2003).

Medications approved for other purposes can help hair grow back in those suffering from alopecia areata, at least temporarily, though none cure the underlying disease. NATIONAL INSTITUTES OF HEALTH, supra. For example, patients may be treated with corticosteroids (e.g., prednisone, dexamethasone, or hydrocortisone) administered orally, topically, or by injection, with oral finasteride, or with a topical solution of minoxidil. Because alopecia areata is an autoimmune disorder, patients are sometimes treated with immunosuppressive compounds as well, (see, e.g., U.S. Pat. No. 5,342,625; U.S. Pat. No. 5,284,826; and U.S. Pat. No. 4,996,193, describing the use of cyclosporin A and related immunosuppressive compounds for hair revitalization, and citing the known use of cyclosporin and related immunosuppressive compounds for hair growth), although such drugs often have significant toxic side effects.

There are two drugs currently approved by the Food & Drug Administration (FDA) for the treatment of male pattern baldness: Rogaine® (topical minoxidil) and Propecia® (oral finasteride). Both were initially used to treat other medical conditions. Minoxidil, a potassium channel agonist that potently induces peripheral vasodilation, was originally used as a treatment for hypertension. The mechanism by which minoxidil induces hair growth is unknown. Dormois et al., “Minoxidil in severe hypertension: value when conventional drugs have failed,” Am. Heart J. 90:360-368 (1975); Messenger, A. G. et al., “Minoxidil: mechanisms of action on hair growth,” Brit. J. Dermatol. 150:186-194 (2004). Finasteride was originally used to treat urinary problems caused by enlargement of the prostate in men (called benign prostatic hyperplasia). It blocks the activity of 5-α-reductase, an enzyme that converts testosterone to dihydrotestosterone (DHT), a more active form of the hormone which has been implicated in miniaturization of hairs, a precursor to catagen. Brown et al., “A current review of medical therapy for benign prostatic hyperplasia,” J. Am. Osteopath. Assoc. 104(S2):S 11-S16 (2004).

Minoxidil and finasteride both stimulate hair regrowth in some patients, but only for the duration of drug use: new hair growth ends and hair loss resumes shortly after the patient stops treatment. After several months' use, minoxidil successfully induces limited hair growth for approximately 1 in 3 patients, and slows hair loss for roughly 9 in 10. Physician's Desk Reference® 2580 (49th ed. 1995). Oral finasteride is generally more effective than topical minoxidil at inducing hair growth, but both treatments are far less than 100% effective. Further hair loss is prevented in most patients treated with finasteride. About half of treated patients achieve some hair regrowth, and approximately one-third of patients experience cosmetically important hair regrowth after two years of continuous use. Foley, P. A., “Recent advances: dermatology,” Brit. Med. J. 320:850-853 (2000).

Both minoxidil and finasteride are sometimes accompanied by a number of potentially serious side-effects. Possible side effects of minoxidil include: scalp itching or rash; headaches; dizziness; decreased libido; elevated heart rate; difficulty breathing; and weight gain. Physician's Desk References 2581 (49th ed. 1995). Possible side effects of finasteride include: skin rash; breast enlargement or tenderness; swelling of lips; testicular pain; decreased libido; decreased volume of ejaculate; and impotence. Physician's Desk Reference® 2067-2069 (58th ed. 2004).

A number of newer methods of treating hair loss employ topical formulations of a variety of compounds, including nucleic acids and various small molecules: (1) a nucleotide sequence encoding the cyclin-dependent kinase inhibitor p21 (U.S. Pat. No. 6,844,326); (2) estrogen receptor antagonists (U.S. Pat. Nos. 6,555,532; 6,204,258; and 5,965,551); (3) a modified, non-immunosuppressive form of Cyclosporin A (U.S. Pat. No. 6,521,595); (4) ketoconazole (U.S. Pat. No. 6,482,826); and (5) compositions containing an aliphatic, alkoxy- or aryl-substituted cyclopropenone (U.S. Pat. No. 4,985,464). None of these treatments has yet received FDA approval.

Some patients seek therapeutic options in addition to drug treatment, including surgical intervention. The most common surgical treatment for hair loss is transplantation, which transfers grafts of skin and hair from the back of the scalp, where hair growth is full, to bald areas. Transplantation may employ mini- or micro-grafts. By this technique, as few as one or two hairs are transplanted with each graft, with 100 or more grafts performed per session. This technique provides a more natural hairline but requires more grafts, and hence more time, than other methods. Bernstein, et al., “The aesthetics of follicular transplantation,” Dermatol. Surg. 23(9):785-799 (1997). A less common surgical treatment for hair loss is scalp reduction, which involves removing areas of bald scalp to bring existing areas of hair growth closer together. Sometimes the skin of the scalp is too tight for this, and alternative treatments must be used.

Autoimmune diseases result from abnormalities in immune cell function or activity which cause inappropriately activated T cells to react against self tissue, thereby triggering production of cytokines or autoantibodies responsible for disease etiology and progression. Autoimmune disorders may be systemic, affecting multiple organs or tissues, or localized, affecting a single organ, organ system or tissue. Limited treatment options focus on: (1) relieving symptoms, whether by administration of analgesics or non-steroidal anti-inflammatory drugs or by surgery; (2) preserving organ function, for example by treating a patient suffering from diabetes mellitus with insulin injections; or (3) targeting disease mechanisms by suppressing the immune system. These treatment options are generally unsatisfactory, because none of them cure the underlying disease, but only ameliorate the symptoms temporarily. In addition, prolonged use of immunosuppressive drugs frequently results in secondary infections, because patients' immune systems cannot repel commonly encountered fungal, bacterial, or viral pathogens.

### SUMMARY OF THE INVENTION

The present invention provides methods for treating hair loss disorders by administration of BMP compositions. The invention further provides methods of treating hair loss disorders by administration of BMP compositions in combination with other compounds used to treat such disorders, including corticosteroids, calcineurin inhibitors, topical minoxidil, and oral finasteride. Some aspects of the invention provide different pharmaceutical formulations of BMP compositions to facilitate different routes of administration. Other aspects of the invention provide different dosage ranges or treatment regimens to treat a wide range of hair loss disorders. Exemplary hair loss disorders that may be treated with the compositions of the invention include alopecia areata, alopecia totalis, alopecia universalis, androgenic alopecia, telogen effluvium, anagen effluvium, and chemotherapy-induced alopecia.

Methods for treating autoimmune disorders by administration of BMP compositions are also provided. The invention further provides methods of treating autoimmune disorders by administration of BMP compositions in combination with other compounds used to treat such disorders, including calcineurin inhibitors and other compounds with known immunosuppressive activity. Some aspects of the invention provide different pharmaceutical formulations of BMP compositions to facilitate different routes of administration. Other aspects of the invention provide different dosage ranges or treatment regimens to treat a wide range of autoimmune disorders. Exemplary autoimmune disorders that may be treated with the compositions of the invention include Crohn's disease, inflammatory bowel disease, multiple sclerosis, psoriasis, rheumatoid arthritis, or systemic lupus erythematosus.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention as claimed.

### BRIEF DESCRIPTION OF THE FIGURES

FIG. 1 shows a photo of patient 602/J-T after treatment with recombinant human BMP-2.

### DETAILED DESCRIPTION OF THE INVENTION

In order that the present invention may be more readily understood, certain terms are first defined. Additional definitions are set forth throughout the detailed description.

As used herein, the term “autoimmune disorder” includes disorders and diseases caused by abnormalities in any immune cell or immune cell function or activity, or any disease or disorder characterized by aberrantly or abnormally elevated immune response. Autoimmune disorders may be systemic, affecting multiple organs or tissues, or localized, affecting a single organ or tissue. Examples of systemic autoimmune diseases include, but are not limited to, rheumatoid arthritis, systemic lupus erythematosus, scleroderma, Sjögren's syndrome, Goodpasture's syndrome, Wegener's granulomatosis, polymyalgia rheumatica, and temporal arteritis or giant cell arteritis. Examples of localized autoimmune diseases include, but are not limited to, alopecia areata, type 1 diabetes mellitus, Hashimoto's thyroiditis, Graves' disease, ulcerative colitis, Crohn's disease, celiac disease, multiple sclerosis, Guillain-Barre syndrome, Addison's disease, primary biliary sclerosis, sclerosing cholangitis, autoimmune hepatitis, and Raynaud's phenomenon.

As used herein, the term “BMP activity” refers to biological activity with the ability to stimulate hair growth or to inhibit immune system activity performed by a BMP composition in a biological system, which may refer to in vitro or in vivo models or therapeutic subjects, depending on the context.

As used herein, the terms “bone morphogenetic protein” or “BMP” refer to any mammalian gene, RNA, or protein of the BMP family of TGF-β proteins, including, but not limited to, BMPs 2-18 and MP52/GDF-5. In particular, a BMP will have an identifying pattern of seven conserved cysteine residues in the mature, carboxy-terminal portion of the protein, as described in Rosen et al., “Bone Morphogenetic Proteins” Principles of Bone Biology 2:919-928 (2002); and Wozney, J. M., “Bone morphogenetic proteins and their gene expression,” CELLULAR AND MOLECULAR BIOLOGY OF BONE 131-167 (Noda, M. ed. 1993). These terms also refer to variants, allelic variants, fragments of, and mutant BMPs, including but not limited to deletion mutants, insertion mutants, and substitution mutants sharing at least 90%, 91%, 92%, 93%, 94%, 95%, 96%, 97%, 98% and 99% amino acid sequence identity with a full-length BMP, or having conservative substitutions at 10%, 9%, 8%, 7%, 6%, 5%, 4%, 3%, 2%, and 1% of amino acid residues, excluding the seven conserved cysteine residues, that retain BMP activity.

As used herein, the term “conservative amino acid substitution” refers to the replacement of a native amino acid by another amino acid of the same or similar physicochemical properties. For example, substitution of one acidic amino acid for another acidic amino acid (e.g., replacing an aspartic acid residue with a glutamic acid residue), one basic amino acid for another basic amino acid (e.g., replacing a histidine residue with a lysine residue), or one polar, uncharged amino acid for another polar, uncharged amino acid (e.g., replacing a serine residue for a threonine residue). The possibility and potential utility of conservative amino acid substitutions in a protein of known sequence is well-understood by one of ordinary skill in the art.

As used herein, the “ED₅₀” (effective dose 50%) is the amount of a compound required to produce a specified effect in 50% of an animal population. As used herein, the “IC₅₀” (inhibitory concentration 50%) is the concentration of a compound which achieves 50% inhibition of its target.

As used herein, the term “immune cell” includes cells that are of hematopoietic origin and that play a role in the immune response, as well as any epithelial or mesenchymal antigen-presenting cells. Immune cells include B lymphocytes, T lymphocytes; natural killer cells and myeloid cells, such as monocytes, macrophages, eosinophils, mast cells, basophils, and granulocytes.

As used herein, the term “immune response” or “immune system activity” includes T and/or B lymphocyte responses, i.e., cellular and/or humoral immune responses. The immune response of a subject can be determined by, for example, assaying antibody production, immune cell proliferation, the release of cytokines, the expression of cell surface markers, cytotoxicity, or by monitoring other indicators of immune system activity.

The term “in combination” as used herein means that a BMP composition containing at least one BMP and a second therapeutic composition are given either simultaneously or sequentially. If given sequentially, at the onset of administration of the second compound, the first of the two compounds may still be detectable at effective concentrations at the site of treatment. It will be understood that, if two therapeutic compositions are administered sequentially, the interval between their administration will be determined by the exigencies of the therapeutic situation and the experience of the treating physician.

The methods disclosed herein can employ either a short, intermediate, or extended course of therapy. As used herein, the language “short course of therapy” includes a therapeutic regimen that is of relatively short duration-relative to the course of the illness being treated. For example, a short course of therapy may last between about one to about four weeks. In contrast, an “intermediate course of therapy” includes a therapeutic regimen that is of longer duration than a short course of therapy. For example, an intermediate course of therapy can last from more than one month to about six months (e.g., between about five to about 26 weeks). An “extended course of therapy” includes those therapeutic regimens that last longer than about six months, e.g., from about seven months on. For example, an extended course of therapy may last from about seven months to as long as the illness persists. The appropriateness of one or more of the courses of therapy described above for any one individual can readily be determined by one of ordinary skill in the art. In addition, the treatment appropriate for a subject may be changed over time as required.

In the course of therapy, doses can be administered early or late. As used herein, “early dosing” includes a therapeutic regimen where BMP compositions are administered to a patient at the onset of disease, e.g., at the onset of clinical symptoms. Alternatively, “late dosing” includes a therapeutic regimen where BMP compositions are administered after disease onset, e.g., after diagnosis or establishment of the disease.

As used herein, the term “TGF-β protein superfamily” refers to a family of structurally-related growth factors. This family-of related growth factors is well-known in the art. Kingsley et al., “The TGF-β superfamily: new members, new receptors, and new genetic tests of function in different organisms,” Genes Dev. 8:133-146 (1994); Hoodless et al., “Mechanism and function of signaling by the TGF-β superfamily,” Curr. Topics Microbiol. Immunol. 228:235-272 (1998). The TGF-β superfamily includes bone morphogenetic proteins (BMPs), activin, inhibin, müllerian-inhibiting substance, glial-derived neurotrophic factor, and a still growing number of growth and differentiation factors (GDFs), such as GDF-8 (myostatin). Piek et al., “Specificity, diversity, and regulation in TGF-β superfamily signaling,” FASEB J. 13:2105-2124 (1999).

As used herein, a “therapeutically effective amount” of at least one BMP is an amount sufficient to stimulate increased hair growth, or to inhibit immune system activity. That amount will depend upon the type and severity of the disorder being treated. The optimal dose of BMP given may even vary in the same patient depending upon the time at which it is administered.

I. Bone Morphogenetic Protein Compositions

According to the present invention, compositions are provided for patients who exhibit signs of the autoimmune disease alopecia areata, other hair loss disorders, or a variety of other autoimmune conditions. Some compositions of the present invention are prepared by mixing at least one active agent with a variety of pharmaceutically acceptable carriers and/or optional excipients to form a liquid, gel, or cream for topical (e.g., transdermal) application. Other compositions of the present invention are prepared by mixing at least one active agent with a variety of pharmaceutically acceptable carriers and optional excipients to form a liquid, gel or solid for administration by injection (e.g., intraosseously, intravenously, parenterally, or percutaneously). In some embodiments of the invention, immune cells may be extracted from a patient suffering from a hair loss disorder or an autoimmune disease, treated with the compositions of the invention, and re-injected into the patient.

A. Bone Morphogenetic Proteins

The active agent may be selected from the family of proteins known as the transforming growth factor-beta (TGF-β) superfamily of proteins, which includes the activins, inhibins, and bone morphogenetic proteins (BMPs). In one embodiment, the active agent includes at least one protein selected from the subclass of proteins known generally as BMPs. The first BMPs (BMPs-1-4) were identified by their ability to induce new bone formation in muscle tissue. Urist et al., “Bone Formation By Autoinduction” Science 150:893-99 (1965). Additional members of the protein subfamily were cloned based on homology with the sequences of BMPs 1-4.

BMPs have been shown to possess a wide range of growth and differentiation activities, including induction of the growth and differentiation of bone, connective, kidney, heart, and neuronal tissues. Rengachary, “Bone Morphogenetic Proteins: Basic Concepts” Neurosurg. Focus 13(6):1-6 (2002). See, for example, descriptions of BMPs in the following publications: BMP-2, BMP-3, BMP-4, BMP-5, BMP-6, and BMP-7 (disclosed, for example, in U.S. Pat. Nos. 5,013,649 (BMP-2 and BMP-4); 5,116,738 (BMP-3); 5,106,748 (BMP-5); 5,187,076 (BMP-6); and 5,141,905 (BMP-7)); BMP-8 (disclosed in PCT WO 91/18098); BMP-9 (disclosed in PCT WO 93/00432); BMP-10 (disclosed in PCT WO 94/26893); BMP-11 (disclosed in PCT WO 94/26892); BMP-12 and BMP-13 (disclosed in PCT WO 95/16035); BMP-15 (disclosed in U.S. Pat. No. 5,635,372); BMP-16 (disclosed in U.S. Pat. No. 6,331,612); MP52/GDF-5 (disclosed in PCT WO 93/16099); and BMP-17 and BMP-18 (disclosed in U.S. Pat. No. 6,027,917). Other TGF-β proteins that may be useful as the active agent in the present invention include Vgr-2, Jones et al., “Isolation of Vgr-2, a novel member of the transforming growth factor-β-related gene family,” Mol. Endocrinol. 6:1961-1968 (1992), and any of the growth and differentiation factors (GDFs), including those described in PCT applications WO 94/15965; WO 94/15949; WO 95/01801; WO 95/01802; WO 94/21681; WO 94/15966; WO 95/10539; WO 96/01845; WO 96/02559 and others.

A subset of BMPs that may be used in certain embodiments of the present invention includes BMP-2, BMP-3, BMP-4, BMP-5, BMP-6, BMP-7, BMP-8, BMP-9, BMP-10, BMP-11, BMP-12 and BMP-13. In an illustrative embodiment, the active agent is BMP-2, the sequence of which is disclosed in U.S. Pat. No. 5,013,649. In another exemplary embodiment, the active agent is BMP-4, the sequence of which is also disclosed in U.S. Pat. No. 5,013,649. In another embodiment, the composition contains two active agents, for example, BMP-2 and BMP-4. Other BMPs and TGF-β proteins known in the art may also be used.

The active agent may be recombinantly produced, or purified from another source. The active agent, if a TGF-β protein such as a BMP, or other dimeric protein, may be homodimeric, or may be heterodimeric with other BMPs (e.g., a heterodimer composed of one monomer each of BMP-2 and BMP-5) or with other members of the TGF-P superfamily, such as activins, inhibins and TGF-μ1 (e.g., a heterodimer composed of one monomer each of a BMP and a related member of the TGF-β superfamily). Examples of such heterodimeric proteins are described, for example in published PCT Patent Application WO 93/09229.

B. Pharmaceutical Formulations and Routes of Administration

A pharmaceutical composition comprising at least one BMP may contain a pharmaceutically acceptable carrier to render the composition suitable for administration to a subject, and a therapeutically effective amount of the active agent. The term subject is intended to include living organisms in which hair growth or an immune response can be elicited, e.g., mammals. Examples of subjects include, but are not limited to, humans, dogs, cats, mice, rats, and transgenic species thereof. The at least one BMP can be administered by various routes well known to persons of ordinary skill in the art, including, but not limited to, parenterally, intravenously, percutaneously, intraosseously, or extra-corporeally.

A pharmaceutical composition for injection could be made up to contain 5 ml sterile buffer containing 0.5% sucrose, 2.5% glycine, 5 mM L-glutamic acid, 5 mM NaCl, and 0.01% polysorbate 80, at pH 4.50, and 5 mg of BMP-2, for a final concentration of 1 mg/ml. A typical pharmaceutical composition for intravenous infusion could be made up to contain 250 ml of sterile Ringer's solution, and 0.05 to 500 mg of at least one BMP. Actual methods of preparing compositions for administration by a variety of routes, including intraosseously, intravenously, parenterally, or percutaneously, will be known or apparent to those skilled in the art and are described in more detail in, for example, PHILADELPHIA COLLEGE OF PHARMACEUTICAL SCIENCES, REMINGTON'S PHARMACEUTICAL SCIENCES (18th ed. 1990).

Solutions or suspensions used for intraosseous, intravenous, parenteral, or percutaneous application typically include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersions. For intravenous administration, suitable carriers include physiological saline, bacteriostatic water, Cremophor® EL (BASF, Parsippany, N.J.) or phosphate buffered saline (PBS). In all cases, the composition must be sterile and should be sufficiently fluid that it is easily injectable via a standard sterile, disposable syringe. It must be stable under the conditions of manufacture and storage and must be preserved against the contaminating action of microorganisms such as bacteria and fungi.

The carrier can be a solvent or dispersion medium containing, for example, water, ethanol, polyol (e.g., glycerol, propylene glycol, or liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol or sorbitol, or sodium chloride in the composition. Prolonged absorption of the injectable compositions can be brought about by including in the composition an agent which delays absorption, for example, aluminum monostearate or gelatin.

In another illustrative embodiment, the compositions that stimulate hair growth or inhibit immune system activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (Mountain View, Calif.) or from other commercial suppliers. Liposomal suspensions may also be used as pharmaceutically acceptable carriers. Such suspensions may be targeted to specific subpopulations of cells or specific subcellular compartments, and can be prepared according to methods known to those skilled in the art, for example, as described in U.S. Pat. No. 4,522,811.

II. Methods of Treatment

A. Conditions to be Treated

Subjects suffering from a wide variety of disorders may be treated by administering the compositions of the invention. For example, the present invention provides methods of treatment for alopecia areata, as well as the related disorders alopecia totalis and alopecia universalis. The invention also provides methods of treatment for other hair loss disorders, including, but not limited to, androgenic alopecia (affecting both male and female patients), telogen effluvium, anagen effluvium, and chemotherapy-induced hair loss. The compositions of the invention may be administered alone, or in combination with additional compounds well known to one of ordinary skill in the art that are used to treat other pathologic conditions or diseases resulting in hair loss.

The present invention further provides methods of treatment for a wide variety of immune system disorders, including, but not limited to, ankylosing spondylitis, antiphospholipid syndrome, Addison's Disease, autoimmune hemolytic anemia, autoimmune hepatitis, autoimmune lymphoproliferative syndrome (ALPS), autoimmune thrombocytopenic purpura (ATP), Behcet's Disease, bulbous pemphigoid, cardiomyopathy, celiac disease, Dermatitis Herpetiformis, chronic fatigue immune dysfunction syndrome (CFIDS), chronic inflammatory demyelinating polyneuropathy (CIDP), cicatricial pemphigoid, systemic sclerosis (CREST Syndrome), cold agglutinin disease, Crohn's Disease, cutaneous vasculitis, Degos' Disease, dermatomyositis, juvenile dermatomyositis, discoid lupus erythematosus, essential mixed cryoglobulinemia, fibromyalgia, Goodpasture's Syndrome, Graves' Disease, Guillain-Barré Syndrome, Hashimoto's Thyroiditis, idiopathic pulmonary fibrosis, idiopathic thrombocytopenia purpura (ITP), immunoglobulin A nephropathy, insulin-dependent diabetes mellitus, juvenile arthritis, Kawasaki's Disease, lichen planus, membranous glomerulonephritis, Ménière's Disease, mixed connective tissue disease, multifocal motor neuropathy, multiple sclerosis, myasthenia gravis, pemphigus vulgaris, pernicious anemia, polyarteritis nodosa, polychondritis, polyglandular syndrome, polymyalgia rheumatica, polymyositis and dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjogren's Syndrome, Stiff-Man Syndrome, systemic lupus erthryomatosus, Takayasu arteritis, temporal arteritis/giant cell arteritis, ulcerative colitis, uveitis, vasculitis, vitiligo, and Wegener's Granulomatosis. The compositions of the invention may be administered alone, or in combination with additional compounds well known to one of ordinary skill in the art to inhibit the immune response.

B. Mechanisms of Action

The BMP compositions of the invention may act in any or all of the following ways to stimulate hair growth and/or to inhibit the immune system. The BMP compositions of the invention may act upon a patient's population of circulating T lymphocytes to alter existing equilibria and cause systemic changes in the immune system, thereby inducing hair growth and/or ameliorating the phenotype of an immune system disorder. The BMP compositions may also be absorbing excess amounts of BMP antagonists, such as anti-BMP antibodies, noggin, chordin, and Cerberus proteins, or other BMP antagonists, to systemically alter BMP metabolism or the activity of the antagonists themselves, thereby inducing hair growth and/or ameliorating the phenotype of an immune system disorder. Finally, the BMP compositions of the invention may increase circulating concentrations of a BMP throughout a subject's body to levels sufficient systemically to stimulate hair growth and/or ameliorate the phenotype of an immune system disorder.

C. Range of Dosage

It is advantageous to formulate compositions administered by injection or inhalation in dosage unit form for ease of administration and uniformity of dosage. Dosage unit form as used herein refers to physically discrete units prepared as unitary dosages for the mammalian subjects to be treated, with each unit containing a predetermined quantity of active compound calculated to produce the desired therapeutic effect in association with the required pharmaceutical carrier.

Data obtained from cell culture assays and animal studies can be used in formulating a range of dosage for use in humans. The dosage may vary within this range depending upon the dosage form employed and the route of administration utilized. For any compound used in the method of the invention, the therapeutically effective dose can be estimated initially from cell culture assays and dosages refined to more accurately determine useful, therapeutically effective doses in humans.

In an exemplary embodiment of the present invention, a therapeutically effective amount of at least one of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, or MP52/GDF-5 may be administered to a subject. In another embodiment, a therapeutically effective-amount of at least one of BMP-2, BMP-4, BMP-5, BMP-6, BMP-7, BMP-10, BMP-12 or BMP-13 may be administered to a subject. In still another embodiment, a therapeutically effective amount of at least one of BMP-2 or BMP-4 may be administered to a patient. In another embodiment, a therapeutically effective amount of both BMP-2 and BMP-4 may be administered to a subject. The optimal dose of at least one BMP given may even vary in the same patient depending upon the time at which it is administered. Generally, the amount of protein to be delivered may range from about 0.05 to about 500 mg, from about 0.5 to about 50 mg, from about 1 to about 25 mg, and from about 5 to about 10 mg per dose. In general, a therapeutically effective amount of at least one BMP would be delivered in solution at a concentration from about 0.001 mg/ml to about 100 mg/ml, from about 0.01 mg/ml to about 50 mg/ml, from about 0.1 mg/ml to about 25 mg/ml, and from about 1 mg/ml to about 5 mg/ml.

The specific dose can be readily calculated by one of ordinary skill in the art, according to the approximate body weight or volume, or body surface area of the patient. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art, based upon standard dose-response studies. It will be understood that the amount of the composition actually administered will be determined by a practitioner, in light of relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells, thereby reducing side effects.

The data obtained from cell culture assays and/or animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the methods of the invention, the therapeutically effective dose can be estimated initially from cell culture assays, and dosages refined without undue experimentation to more accurately determine useful doses in humans. For example, plasma levels of the active agent may be measured by high performance liquid chromatography.

Thus, the dosage of any of the subject agents, can be easily determined by one of ordinary skill in the art. The dose may vary depending on the age, health and weight of the recipient, the extent of disease, kind of concurrent treatment, if any, frequency of treatment and the nature of the effect desired. It should be noted that the dose of BMP given to one subject may vary during the course of the treatment.

D. Timing of Treatment

The skilled artisan will appreciate that certain factors may influence the dosage required to effectively treat a subject, including but not limited to the severity of the-disease or disorder, previous treatments, the general health and/or age of the subject, and other diseases present. Moreover, treatment of a subject can include a single treatment or can include a series of treatments administered daily, weekly, monthly, bi-monthly, tri-monthly, bi-annually, and annually. In some cases it may be necessary to continue treatment for an indefinite period, or for as long as hair growth or a decreased immune response is desired. It will also be appreciated that the therapeutically effective dosage of a BMP used to stimulate hair growth or to inhibit the immune system may increase or decrease over the course of a particular treatment. Changes in dosage may result from relapse of the disease or worsening of symptoms.

The present compositions comprising BMPs can be administered for prophylactic and/or therapeutic treatments. In prophylactic application, compositions can be administered to a patient especially susceptible or otherwise expecting to suffer hair loss or increased immune system activity, for example, resulting from a course of chemotherapy. In therapeutic application, compositions can be administered to a patient already suffering from a disease, for example, alopecia areata, alopecia totalis, alopecia universalis, androgenic alopecia, rheumatoid arthritis or other autoimmune disease, in an amount sufficient to ameliorate the symptoms of the disease and associated complications. Single or multiple administrations of the compositions can be carried out with dose levels and pattern sufficient effectively to treat the patient being selected by the treating physician.

E. Combination Therapy for Stimulating Hair Growth

Administration of the compositions of the invention as described herein may be as a therapeutically effective formulation containing a therapeutically active amount of at least one BMP alone or in combination with any other therapeutic composition or molecule. The combination therapy is useful for treating pathological conditions or disorders resulting in hair loss. The term “in combination” in this context means that the BMP composition and a second therapeutic composition are given either simultaneously or sequentially. If given sequentially, at the onset of administration of the second compound, the first of the two compounds may still be detectable at effective concentrations at the site of treatment.

For example, the combination therapy may include at least one BMP composition co-formulated with, and/or co-administered with, at least one additional therapeutic agent for stimulating hair growth. The additional agents may include at least one of the following, administered either orally, topically, by inhalation or by injection: (1) corticosteroids, such as prednisone, dexamethasone, or hydrocortisone; (2) calcineurin inhibitors known to have immunosuppressive activity, such as cyclosporin A, pimecrolimus or tacrolimus; (3) minoxidil; or (4) finasteride. Such combination therapies may advantageously utilize lower dosages of the administered therapeutic agents, thus avoiding possible toxicities or complications associated with the various monotherapies alone. Moreover, the additional therapeutic agents disclosed herein act upon metabolic pathways other than those regulating BMP metabolism, and thus are expected to enhance and/or synergize with the effects of the BMP compositions.

Administration of a therapeutically active amount of the compositions of the present invention is defined as an amount effective, at dosages and for periods of time necessary to achieve the desired result. For example, a therapeutically active amount of at least one BMP, a corticosteroid, a calcineurin inhibitor, minoxidil, or finasteride may vary according to factors such as the disease state, age, sex, and weight of the individual, and the ability of the compound to elicit a desired response in the individual. A dosage regime may be adjusted to provide the optimum therapeutic response. For example, several divided doses may be administered daily, a single dose may be administered daily, weekly, monthly or at longer intervals, or the dose may be proportionally reduced as indicated by the exigencies of the therapeutic situation.

F. Combination Therapy for Inhibiting Immune System Activity

Administration of the compositions of the invention as described herein can be as a therapeutically effective formulation containing a therapeutically active amount of at least one BMP alone or in combination with any other therapeutic composition or molecule. The combination therapy is useful for treating pathological conditions or disorders of the immune system, especially those characterized by aberrant autoimmune activity. The term “in combination” in this context means that the BMP composition and a second therapeutic composition are given either simultaneously or sequentially. If given sequentially, at the onset of administration of the second compound, the first of the two compounds may still be detectable at effective concentrations at the site of treatment.

For example, the combination therapy can include at least one BMP composition co-formulated with, and/or co-administered with, at least one additional therapeutic agent for reducing immune system activity. The additional agents may include at least one of the following, administered either orally, topically, by inhalations or by injection: (1) calcineurin inhibitors known to have immunosuppressive activity, such as cyclosporin A, pimecrolimus or tacrolimus; or (2) other compounds with immunosuppressive activity, for example, azathioprine, mycophenolate mofetil, rapamycin or rapamycin analogs (e.g., CCI-779).

Administration of a therapeutically active amount of the compositions of the present invention is defined as an amount effective, at dosages and for periods of time necessary to achieve the desired result. For example, a therapeutically active amount of at least one BMP, a calcineurin inhibitor, or another immunosuppressive compound may vary according to factors such as the disease state, age, sex, and weight of the individual, and the ability of the compound to elicit a desired response in the individual. A dosage regime may be adjusted to provide the optimum therapeutic response. For example, several divided doses may be administered daily, a single dose may be administered daily, weekly, monthly or at longer intervals, or the dose may be proportionally reduced as indicated by the exigencies of the therapeutic situation.

The BMP compositions disclosed herein can be used in combination with other therapeutic agents to treat specific immune disorders as discussed in further detail below.

Non-limiting examples of agents for treating arthritic disorders (e.g., rheumatoid arthritis, inflammatory arthritis, juvenile rheumatoid arthritis, osteoarthritis and psoriatic arthritis), with which a BMP composition may be combined include at least one of the following: TNF antagonists (such as anti-TNF antibodies); soluble fragments of TNF receptors (e.g., human p55 and p75) and derivatives thereof (such as p55 kdTNFR-IgG (55 kD TNF receptor-IgG fusion protein, Lenercept®) and 75 kd TNFR-IgG (75 kD TNF receptor-IgG fusion protein, Enbrel®)); TNF enzyme antagonists (such as TNFα converting enzyme, or TACE, inhibitors); antagonists of IL-12, IL-15, IL-17, IL-18, and IL-22; T cell and B cell depleting agents (such as anti-CD4 or anti-CD22 antibodies); small molecule inhibitors (such as methotrexate and leflunomide); COX-2 and cPLA2 inhibitors; non-steroidal anti-inflammatory drugs (NSAIDs); p38, TPL-2, Mk-2, and NFκB inhibitors; receptor for advanced glycation end products (RAGE) or soluble RAGE; P-selectin or PSGL-1 inhibitors (such as small molecule inhibitors and antibodies thereto); estrogen receptor β (ERβ) agonists, and ERβ-NFκB antagonists.

Non-limiting examples of agents for treating multiple sclerosis with which a BMP composition may be combined include interferon-β (for example, IFNP-1α and IFNβ-1b), copaxone, corticosteroids, IL-I inhibitors, TNF inhibitors, antibodies to CD40 ligand, antibodies to CD80, and IL-12 antagonists.

Non-limiting examples of agents for treating inflammatory bowel disease or Crohn's disease with which BMP compositions may be combined include budenoside; epidermal growth factor; corticosteroids; cyclosporin; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies; growth factors; elastase inhibitors; pyridinyl-imidazole compounds; TNF antagonists as described herein; IL-4, IL-10, IL-13, and/or TGFb or agonists thereof (e.g., agonist antibodies); IL-11; glucuronide- or dextran-conjugated prodrugs of prednisone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

G. Methods for Evaluating the Ability of Pharmaceutical Compositions to Stimulate Hair Growth

The ability of the compositions of the invention as described herein to stimulate hair growth in a subject can be determined by, for example, assaying expression of hair- or hair follicle-specific markers of cell growth and differentiation, including keratin 6, keratin 16, keratin 17, β-catenin, and trichohyalin, by measuring uptake of bromodeoxyuridine in dividing hair follicle stem cells or by measuring alkaline phosphatase activity in dermal papilla cells. The compositions of the invention may also be tested for their ability to stimulate hair growth in vitro on cultures of matrix keratinocytes, on dissected human hair follicles, or on whole skin explants grown on collagen sponges for the ability to induce cell and/or hair growth and proliferation. Alternatively, the compositions of the invention may be tested for their ability to stimulate hair growth on mice after clipping or shaving a portion of their body hair, as described in Example 2.

It will be understood that each of the various hair loss disorders treatable by the methods of the present invention has a characteristic molecular phenotype measurable using techniques well known to persons of ordinary skill in the art. That phenotype may be characterized by increased or decreased levels of circulating auto-antibodies, increased or decreased levels of T lymphocytes or subpopulations thereof, altered expression levels or patterns of hair follicle-specific cell surface antigens, or by other molecular markers of cell growth and proliferation. These phenotypes may be assayed before and after treatment with the compositions of the invention using a variety of routine methods, including flow cytometry, immunohistochemistry, enzyme-linked immunosorbent assays (ELISA), Western blotting, reverse transcription-polymerase chain reaction (RT-PCR), and/or transcription profiling.

H. Methods for Evaluating the Ability of Pharmaceutical Compositions to Inhibit Immune System Activity

The ability of the compositions of the invention as described herein to inhibit immune system activity in a subject can be determined by, for example, assaying serum antibody levels, immune cell proliferation or markers thereof, the release of cytokines, the expression of cell surface markers, cytotoxicity, or by monitoring other indicators of immune system activity. It will be understood that each of the various autoimmune disorders treatable by the methods of the present invention has a characteristic molecular phenotype measurable using techniques well known to persons of ordinary skill in the art. That phenotype may be characterized by increased or decreased levels of circulating auto-antibodies, increased or decreased levels of T or B lymphocytes or subpopulations thereof,.altered expression levels or patterns of T lymphocyte- or B lymphocyte-specific cell surface antigens, or by other molecular markers of cell growth and proliferation. These phenotypes may be assayed before and after treatment with the compositions of the invention using a variety of routine methods, including flow cytometry, immunohistochemistry, enzyme-linked immunosorbent assays (ELISA), Western blotting, reverse transcription-polymerase chain reaction (RT-PCR), and/or transcription profiling.

For example, patients suffering from systemic lupus erythematosus (SLE) frequently have high serum levels of antibodies directed to DNA, or to ribonucleoproteins (RNPs). The efficacy of the methods f-treatment described herein may be evaluated by quantifying the amount of circulating anti-DNA or anti-RNP antibodies in the serum of a patient suffering from SLE before and after treatment using, for example, a sandwich ELISA. A method of treatment as described herein would be deemed effective if it resulted in a decrease in circulating levels of anti-DNA or anti-RNP auto-antibodies compared to pre-treatment levels.

### EXAMPLES

### Example 1

### Treating a 21-Year-Old Man Having Alopecia Universalis with BMP-2

A 21-year-old man presented to the dermatology department of Norfolk & Norwich University in March, 2004, with alopecia. The patient had first noticed patches of scalp hair loss 2 years previously. Hair loss progressed rapidly from this first observation, and within 6 months the patient had lost every hair from all body sites. The patient's past medical history includes asthma and childhood eczema, as well as a family history of alopecia areata (both his maternal grandmother and one cousin had the disease). Examination confirmed the diagnosis of alopecia universalis in an otherwise fit young man. Routine blood tests, including full blood count, urea and electrolytes, liver function tests and auto-immune serology, were all within normal range.

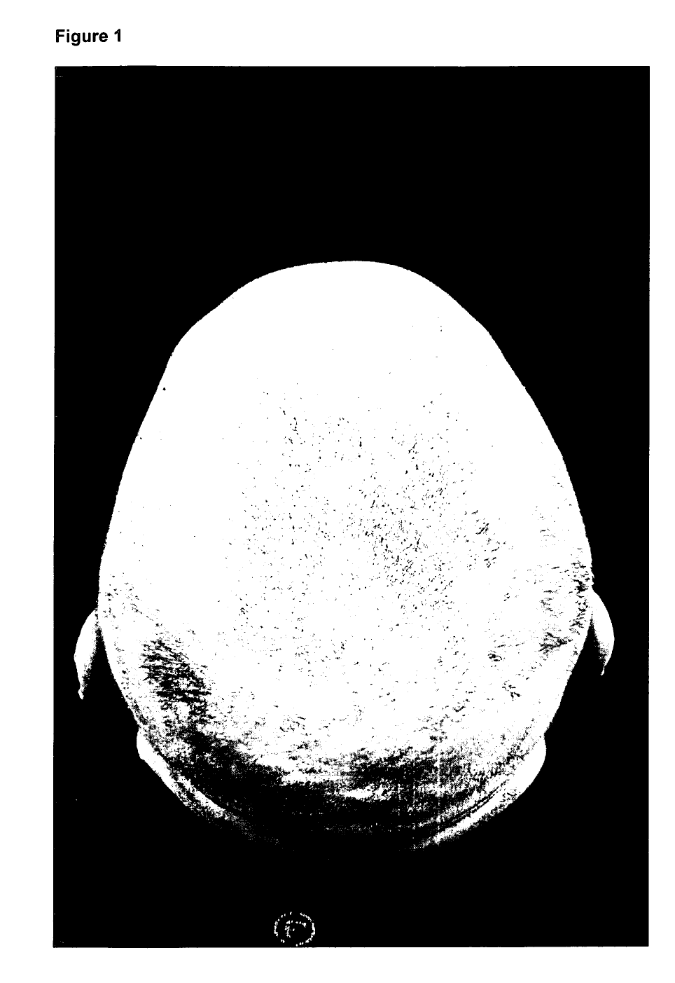

One month later, the patient fractured his left tibia while playing soccer. His tibia was set with a Russell-Taylor tibial nail inserted. Six days later, on Apr. 25, 2004, the patient consented to participate in a trial using recombinant human BMP-2 (rhBMP-2), potentially to speed the healing of the tibial fracture. The closed tibial fracture was injected with 5 ml of rhBMP-2 at 1.0 mg/ml in a calcium phosphate matrix, and the patient was subsequently discharged from hospital. Six weeks later, the patient noticed hair re-growth initially on his scalp (see FIG. 1). Hair growth became more extensive, with hair eventually returning to eyebrows, axillae, arms and pubic area. This hair re-growth was maintained for six months following the treatment with rhBMP-2. The tibial fracture has also healed satisfactorily.

### Example 2

### BMP-2 and BMP-4 Stimulate Hair Growth in C57BL/6 Mice

The ability of the compositions of the invention to stimulate hair growth is evaluated using a population of 45 sex- and age-matched C57BL/6 mice, separated into three groups of 15: two experimental groups and one control group. Hair from a roughly 4-cm² region of dorsal skin on all animals is clipped to 0.1 mm in length. The experiment will test a range of five different doses of BMP-2 and BMP-4: 500 μg, 1 μg, 2.5 μg, 5 μg, 10 μg. Each single use dose is prepared in an isotonic saline solution at BMP-2 or BMP-4 concentrations sufficient to allow each dose to be administered in a total volume of 25 μl.

Three animals in the first experimental group are injected with 25 μl each of isotonic saline containing 500 ng of BMP-2 (20 ng/μl), three are injected with 25 μl each of isotonic saline containing 1 μg of BMP-2 (40 ng/μl), three are injected with 25 μl each of isotonic saline containing 2.5 μg of BMP-2 (100 ng/μl), three are injected with 25 μl each of isotonic saline containing 5 μg of BMP-2 (200 ng/μl), and three are injected with 25 μl each of isotonic saline containing 10 μg of BMP-2 (400 ng/μl). Three animals in the second experimental group are injected with 25 μl each of isotonic saline containing 500 ng of BMP-4 (20 ng/μl), three are injected with 25 μl each of isotonic saline containing 1 μg of BMP-4 (40 ng/μl), three are injected with 25 μl each of isotonic saline containing 2.5 μg of BMP-4 (100 ng/μl), three are injected with 25 μl each of isotonic saline containing 5 μg of BMP-4 (200 ng/μl), and three are injected with 25 μl each of isotonic saline containing 10 μg of BMP-4 (400 ng/μl).

Similarly, animals in the control group are separated into five groups of three. The first group of three is injected with 25 μl each of an isotonic saline solution containing 500 ng bovine serum albumin (BSA) (20 ng/μl), the second group is injected with 25 μl each of an isotonic saline solution containing 1 μg BSA (40 ng/μl), the third group is injected with 25 μl each of an isotonic saline solution containing 2.5 μg BSA (100 ng/μl), the fourth group is injected with 25 μl each of an isotonic saline solution containing 5 μg BSA (200 ng/μl), and the fifth group is injected with 25 μl each of an isotonic saline solution containing 10 μg BSA (400 ng/μl).

All mice in both groups are examined twice daily for hair growth for a period of twenty-one days. It is expected that hair regrowth will be faster in mice receiving BMP-2 or BMP-4 than in mice receiving only BSA. It is further expected that the stimulation of hair regrowth will correlate with the amount of BMP-2 or BMP-4 received. Thus it is expected that the difference in hair regrowth between the control group and the experimental groups will be most significant and noticeable for those mice receiving either 5 μg (200 ng/μl) or 10 μg (400 ng/μl) of BMP-2 or BMP-4. Similarly, it is expected that the difference in hair regrowth will be least significant and noticeable for those mice receiving either 500 ng (20 ng/μl) or 1 μg (40 ng/μl) of BMP-2 or BMP-4.

After twenty-one days, the mice are euthanized and skin sections are prepared for histological and immunohistochemical analysis of markers of hair follicle growth and development, including bromodeoxyuridine (BrdU) labelling, trichohyalin and hair keratin expression, and alkaline phosphatase activity in dermal papilla cells. It is expected that BrdU labeling, a marker of proliferation in epidermal stem cells of the hair follicle, will increase with increasing doses of BMP-2 or BMP-4. Similarly, expression of hair follicle-specific keratins and trichohyalin is expected to increase with increasing doses of BMP-2 or BMP-4, as measured by real time RT-PCR and transcription profiling.

### Example 3

### Treating Patients Suffering from Androgenic Alopecia with BMP-2

In one study, a cohort of normal, healthy men suffering from androgenic alopecia is identified. Initially, all subjects are evaluated by routine blood tests, including full blood count, urea and electrolytes, blood pressure and liver function tests as well as auto-immune serology. In accord with generally accepted practice for the conduct of a standard randomized, double-blind clinical trial, the patients are given identification numbers separating them into an experimental group that will receive BMP-2, and a control group that will receive a placebo. The trial will test three treatment regimens, ranging from a single injected dose of 5 mg, to three bimonthly injections of 5 mg each, to six consecutive monthly injections of 5 mg each. Whether administered alone or in combination (see below), the BMP-2 will be administered by a single injection of 5 ml of BMP-2 prepared at a concentration of 1 mg/ml, formulated in an appropriate sterile pharmaceutical solution. Outcome assessments are based on weekly visual examinations of hair growth and density, as well as on levels of markers of hair follicle growth and development measured in tissue taken from scalp biopsies before treatment, and three, six and twelve months after treatment. It is expected that increased hair growth and density, as well as increased expression of hair follicle-specific intermediate filament proteins such as keratins and trichohyalin, will correlate: directly with increased doses of BMP-2.

In another study, a second cohort of normal, healthy men suffering from androgenic alopecia is identified. As before, all subjects are evaluated by routine blood tests, including full blood count, urea and electrolytes, blood pressure and liver function tests as well as auto-immune serology. In accord with generally accepted practice for the conduct of a standard randomized, double-blind clinical trial, the patients are given identification numbers separating them into an experimental group that will receive BMP-2, and a control group that will receive a placebo. The trial will test four combination therapies, each including three bi-monthly injections of 5 mg BMP-2 combined with (1) prednisone, (2) cyclosporin A, (3) a 5% topical solution of minoxidil, or (4) oral finasteride. Doses of the latter four medications are administered in accord with accepted clinical practice as determined by the physicians overseeing the trial. Outcome assessments are based on weekly visual examinations of hair growth and density, as well as on levels of markers of hair follicle growth and development measured in tissue taken from scalp biopsies before treatment, and three, six and twelve months after treatment. It is expected that increased hair growth and density, as well as increased expression of hair follicle-specific intermediate filament proteins such as keratins and trichohyalin, will correlate directly with increased doses of BMP-2 in combination with corticosteroids, calcineurin inhibitors, minoxidil or finasteride.

### Example 4

### Treating Patients Suffering from Systemic Lupus Erythematosus with BMP-2

In one study, a cohort of normal, otherwise healthy female patients suffering from systemic lupus erythematosus (SLE) is identified. Initially, all subjects are evaluated by routine blood tests, including full blood count, urea and electrolytes, blood pressure and liver function tests as well as auto-immune serology. In accord with: generally accepted practice for the conduct of a standard randomized, double-blind clinical trial, the patients are given identification numbers separating them into an experimental group that will receive BMP-2, and a control group that will receive a placebo. The trial will test three treatment regimens, ranging from a single injected dose of 5 mg, to three bimonthly injections of 5 mg each, to six consecutive monthly injections of 5 mg each. Whether administered alone or in combination (see below), the BMP-2 will be administered by a single injection of 5 ml of BMP-2 prepared at a concentration of 1 mg/ml, formulated in an appropriate sterile pharmaceutical solution. Outcome assessments are based on circulating serum concentrations of antibodies characteristic of SLE, including anti-dsDNA (directed against double stranded DNA), anti-Sm (directed against six different small, nuclear RNA molecules), and anti-RNP (directed against U1 RNA), as measured by conventional ELISA before treatment, and weekly for twelve months after treatment commences. It is expected that a decrease in serum levels of some or all of these auto-antibodies will correlate with increasing doses of BMP-2.

In another study, a second cohort of normal, healthy women suffering from SLE is identified. As before, all subjects are evaluated by routine blood tests, including full blood count, urea and electrolytes, blood pressure and liver function tests as well as auto-immune serology. In accord with generally accepted practice for the conduct of a standard randomized, double-blind clinical trial, the patients are given identification numbers separating them into an experimental group that will receive BMP-2 in combination with one of two types of immunosuppressive compounds, and a control group that will receive a placebo. The trial will test two combination therapies, each including three bi-monthly injections of 5 mg BMP-2 combined with (1) cyclosporin A, or (2) rapamycin. Doses of the latter two medications are administered in accord with accepted clinical practice as determined by the physicians overseeing the trial. Outcome assessments are based on circulating serum concentrations of antibodies characteristic of SLE, including anti-dsDNA (directed against double stranded DNA), anti-Sm (directed against six different small, nuclear RNA molecules), and anti-RNP (directed against U1 RNA), as measured by conventional ELISA before treatment, and weekly for twelve months after treatment commences. It is expected that a decrease in serum levels of some or all of these auto-antibodies will correlate with increasing doses of BMP-2 in combination with a calcineurin inhibitor or a known immunosuppressor.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

### REFERENCES

Bernstein, et al., “The aesthetics of follicular transplantation,” Dermatol. Surg. 23(9):785-799 (1997).

Botchkarev, V. A., “Bone morphogenetic proteins and their antagonists in skin and hair follicle biology,” J. Invest Dermatol. 120(1):36-47 (2002).

Botchkarev et al., “BMP signaling in the control of skin development and hair follicle growth,” Differentiation 72:512-526 (2004).

Brown et al., “A current review of medical therapy for benign prostatic hyperplasia,” J. Am. Osteopath. Assoc. 104(S2):S11-S16 (2004).

Cotsarelis et al., “Towards a molecular understanding of hair loss and its treatment,” TRENDS in Mol. Med. 7(7):293-301 (2001).

Dormois et al., “Minoxidil in severe hypertension: value when conventional drugs have failed,” Am. Heart J. 90:360-368 (1975).

Foley, P. A., “Recent advances: dermatology,” Brit. Med. J. 320:850-853 (2000).

Hull et al., “Guidelines for the management of alopecia areata,” Br. J. Dermatol. 149:692-699 (2003).

Hoodless et al., “Mechanism and function of signaling by the TGF-β superfamily,” Curr. Topics Microbiol. Immunol. 228:235-272 (1998).

Jones et al., “Isolation of Vgr-2, a novel member of the transforming growth factor-β-related gene family,” Mol. Endocrinol. 6:1961-1968 (1992).

Kalish, et al., “Alopecia areata: autoimmunity—the evidence is compelling,” J. Invest. Dermatol. 8(2):164-167 (2003).

Kingsley et al., “The TGF-β superfamily: new members, new receptors, and new genetic tests of function in different organisms,” Genes Dev. 8:133-146 (1994).

MacDonald, N., “Alopecia areata: identification and current treatment approaches,” Dermatol. Nurs. 11:356-359 (1999).

Messenger, A. G. et al., “Minoxidil: mechanisms of action on hair growth,” Brit. J. Dermatol. 150:186-194 (2004).

NATIONAL INSTITUTES OF HEALTH, Questions & Answers About Alopecia Areata (2003).

Paus, et al., “The biology of hair follicles,” N. Engl. J. Med. 341(7):491-497 (1999).

Physician's Desk Reference® (49th ed. 1995).

Physician's Desk Reference® (58th ed. 2004).

Piek et al., “Specificity, diversity, and regulation in TGF-β superfamily signaling,” FASEB J. 13:2105-2124 (1999).

Philadelphia College of Pharmaceutical Sciences, Remington's Pharmaceutical Sciences (18th ed. 1990).

Rengachary, “Bone Morphogenetic Proteins: Basic Concepts” Neurosurg. Focus 13(6):1-6 (2002).

Rosen et al., “Bone Morphogenetic Proteins” Principles of Bone Biology 2:919-928 (2002).

Stenn et al., “Controls of Hair Follicle Cycling,” Physiol. Rev. 81:449-494 (2001).

Urist et al., “Bone Formation By Autoinduction” Science 150:893-99 (1965).

Vierhapper et al., “Production rates of testosterone and of dihydrotestosterone in female pattern hair loss,” Metabolism 52(7):927-929 (2003).

Wozney, J. M., “Bone morphogenetic-proteins and their gene expression,” CELLULAR AND MOLECULAR BIOLOGY OF BONE 131-167 (Noda, M. ed. 1993).

What is claimed is:

1. A method of treating a hair loss disorder comprising:
(a) identifying a patient suffering from a hair loss disorder;
(b) administering a composition comprising a therapeutically effective amount of at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5; and
(c) allowing the at least one active agent to promote hair growth.

2. The method of claim 1, wherein the hair loss disorder is chosen from the group comprising: alopecia areata, alopecia totalis, alopecia universalis, androgenic alopecia, telogen effluvium, anagen effluvium, and chemotherapy-induced alopecia.

3. The method of claim 1, wherein the composition comprises a therapeutically effective amount of BMP-2.

4. The method of claim 1, wherein the composition comprises a therapeutically effective amount of BMP-4.

5. The method of claim 1, wherein the composition comprises a therapeutically effective amount of BMP-2 and a therapeutically effective amount of BMP-4.

6. The method of claim 1, wherein the subject is a mammal.

7. The method of claim 1, wherein the subject is a human.

8. The method of claim 1, wherein the composition is administered by injection, intraosseously, intravenously, parenterally, percutaneously or extra-corporeally.

9. The method of claim 1, wherein the therapeutically effective amount of at least one BMP is chosen from about 0.05 to about 500 mg, from about 0.5 to about 50 mg, from about 1 to about 25 mg, and from about 5 to about 10 mg.

10. The method of claim 1, wherein the therapeutically effective amount of at least one BMP is administered at a concentration chosen from about 0.001 mg/ml to about 100 mg/ml, from about 0.01 mg/ml to about 50 mg/ml, from about 0.1 mg/ml to about 25 mg/ml, and from about 1 mg/ml to about 5 mg/ml.

11. The method of claim 1, wherein the therapeutically effective amount of at least one BMP is administered to the subject at intervals chosen from: daily, weekly, monthly, bimonthly, tri-monthly, biannually, and annually.

12. The method of claim 1, wherein the therapeutically effective amount of at least one BMP is administered to the subject for a period of time chosen from: about 1 to about 4 weeks, about 5 to about 24 weeks, about 25 to about 52 weeks, about 1 to about 2 years, about 2 to about 5 years, about 5 to about 10 years and about 10 to about 20 years.

13. The method of claim 2, wherein the hair loss disorder is alopecia areata, alopecia totalis, or alopecia universalis.

14. A method of treating a hair loss disorder comprising:
(a) identifying a patient suffering from a hair loss disorder;
(b) administering a composition comprising a therapeutically effective amount of at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5;
(c) administering a therapeutically effective amount of at least one compound chosen from the group consisting of prednisone, dexamethisone, hydrocortisone, cyclosporin A, pimecrolimus, tacrolimus, minoxidil, and finasteride; and
(d) allowing the active agents to promote hair growth.

15. A method of treating an autoimmune disorder comprising:
(a) identifying a patient suffering from an autoimmune disorder;
(b) administering a composition comprising a therapeutically effective amount of at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5; and
(c) allowing the at least one BMP to inhibit immune system activity.

16. The method of claim 15, wherein the autoimmune disorder is chosen from the group comprising: ankylosing spondylitis, antiphospholipid syndrome, Addison's Disease, autoimmune hemolytic anemia, autoimmune hepatitis, autoimmune lymphoproliferative syndrome (ALPS), autoimmune thrombocytopenic purpura (ATP), Behcet's Disease, bulbous pemphigoid, cardiomyopathy, celiac disease, Dermatitis Herpetiformis, chronic fatigue immune dysfunction syndrome (CFIDS), chronic inflammatory demyelinating polyneuropathy (CIDP), cicatricial pemphigoid, systemic sclerosis (CREST Syndrome), cold agglutinin disease, Crohn's Disease, cutaneous vasculitis, Degos' Disease, dermatomyositis, juvenile dermatbmyositis, discoid lupus erythematosus, essential mixed cryoglobulinemia, fibromyalgia, Goodpasture's Syndrome, Graves' Disease, Guillain-Barre Syndrome, Hashimoto's Thyroiditis, idiopathic pulmonary fibrosis, idiopathic thrombocytopenia purpura (ITP), immunoglobulin A nephropathy, inflammatory arthritis, insulin-dependent diabetes mellitus, juvenile arthritis, Kawasaki's Disease, lichen planus, membranous glomerulonephritis, Ménière's Disease, mixed connective tissue disease, multifocal motor neuropathy, multiple sclerosis, myasthenia gravis, osteoarthritis, pemphigus vulgaris, pernicious anemia, polyarteritis nodosa, polychondritis, polyglandular syndrome, polymyalgia rheumatica, polymyositis and dermatomyositis, primary agammaglobulinemia, primary biliary cirrhosis, psoriasis, psoriatic arthritis, Raynaud's Phenomenon, Reiter's Syndrome, rheumatic fever, rheumatoid arthritis, sarcoidosis, scleroderma, Sjögren's Syndrome, Stiff-Man Syndrome, systemic lupus erthryomatosus, Takayasu arteritis, temporal arteritis/giant cell arteritis, ulcerative colitis, uveitis, vasculitis, vitiligo, and Wegener's Granulomatosis.

17. The method of claim 15, wherein the composition comprises a therapeutically effective amount of BMP-2.

18. The method of claim 15, wherein the composition comprises a therapeutically effective amount of BMP-4.

19. The method of claim 15, wherein the composition comprises a therapeutically effective amount of BMP-2 and a therapeutically effective amount of BMP-4.

20. The method of claim 15, wherein the subject is a mammal.

21. The method of claim 15, wherein the subject is a human.

22. The method of claim 15, wherein the composition is administered by injection, intraosseously, intravenously, parenterally, percutaneously or extra-corporeally.

23. The method of claim 15, wherein the therapeutically effective amount of at least one BMP is chosen from about 0.05 to about 500 mg, from about 0.5 to about 50 mg, from about 1 to about 25 mg, and from about 5 to about 10 mg.

24. The method of claim 15, wherein the therapeutically effective amount of at least one BMP is administered at a concentration chosen from about 0.001 mg/ml to about 100 mg/ml, from about 0.01 mg/ml to about 50 mg/ml, from about 0.1 mg/ml to about 25 mg/ml, and from about 1 mg/ml to about 5 mg/ml.

25. The method of claim 15, wherein the therapeutically effective amount of at least one BMP is administered to the subject at intervals chosen from: daily, weekly, monthly, bimonthly, tri-monthly, biannually, and annually.

26. The method of claim 15, wherein the therapeutically effective amount of at least one BMP is administered to the subject for a period of time chosen from: about 1 to about 4 weeks, about 5 to about 24 weeks, about 25 to about 52 weeks, about 1 to about 2 years, about 2 to about 5 years, about 5 to about 10 years and about 10 to about 20 years.

27. The method of claim 16, wherein the autoimmune disorder is Crohn's disease, inflammatory bowel disease, multiple sclerosis, psoriasis, rheumatoid arthritis, or systemic lupus erythematosus.

28. A method of treating an autoimmune disorder comprising:
(a) identifying a patient suffering from an autoimmune disorder;
(b) administering a composition comprising a therapeutically effective amount of at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5;
(c) administering a therapeutically effective amount of at least one compound chosen from the group consisting of cyclosporin A, pimecrolimus, tacrolimus, azathioprine, mycophenolate mofetil, rapamycin, CCI-779, methotrexate, leflunomide, interferon-β, copaxone, budenoside, epidermal growth factor, sulfasalazine, 6-mercaptopurine, azathioprine, metronidazole, mesalamine, olsalazine, ciprofloxacin, and lignocaine; and
(d) allowing the BMPs to inhibit immune system activity.

29. A pharmaceutical composition comprising:
(a) at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5;
(b) a sterile solution of 0.5% sucrose, 2.5% glycine, 5 mM L-glutamic acid, 5 mM NaCl, and 0.01% polysorbate 80, at pH 4.50.

30. A pharmaceutical composition for promoting hair growth comprising:
(a) at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5; and
(b) at least one compound chosen from the group consisting of prednisone, dexamethisone, hydrocortisone, cyclosporin A, pimecrolimus, tacrolimus, minoxidil, and finasteride.

31. A pharmaceutical composition for inhibiting immune system activity comprising:
(a) at least one BMP chosen from the group consisting of BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, and MP52/GDF-5,
(b) and at least one compound chosen from the group consisting of cyclosporin A, pimecrolimus, tacrolimus, azathioprine, mycophenolate mofetil, rapamycin, CCI-779, methotrexate, leflunomide, interferon-β, copaxone, budenoside, epidermal growth factor, sulfasalazine, 6-mercaptopurine, azathioprine, metronidazole, mesalamine, olsalazine, ciprofloxacin, and lignocaine.
